(19) United States
(12) Patent Application Publication
(10) Pub. No.: US 2026/0151206 A1
(43) Pub. Date: Jun. 4, 2026

(54) OUTER PACKAGING FOR MEDICAL PRODUCTS AND METHOD FOR PACKAGING MEDICAL PRODUCTS

(71) Applicant: Aesculap AG, Tuttlingen (DE)

(72) Inventors: Florian Etzel, Winnenden (DE); Stefanie Weber, Villingen-Schwenningen (DE)

(21) Appl. No.: 18/864,307

(22) PCT Filed: May 9, 2023
(86) PCT No.: PCT/EP2023/062214

§ 371 Date: Nov. 8, 2024

(30) Foreign Application Priority Data
May 9, 2022 (DE) 10 2022 111 498.8

(51) Int. Cl. A61B 50/30

(52) U.S. Cl. CPC A61B 50/30

(57) ABSTRACT

A foldable outer packaging for a medical product includes a base panel subdivided into three segments by two flap fold lines that are spaced apart in parallel, a hinged panel coupled to the base panel by a hinge fold line oriented perpendicularly to the flap fold lines and also subdividing the hinged panel into three segments by the flap fold lines that extend in a straight line into the hinged panel, and a window-like cut-out or opening in the central one of the three segments, and a side panel coupled to the hinged panel by a tilting fold line oriented parallel to the hinge fold line and subdividing the side panel into three segments by the flap fold lines that further extend in a straight line into the side panel. The foldable outer packaging can be used to make outer packaging for a medical product.

### CROSS-REFERENCE TO RELATED APPLICATIONS

This application is the United States national stage entry of International Application No. PCT/EP2023/062214, filed on May 9, 2023, and claims priority to German Application No. 10 2022 111 498.8, filed on May 9, 2022. The contents of International Application No. PCT/EP2023/062214 and German Application No. 10 2022 111 498.8 are incorporated by reference herein in their entireties.

### FIELD

The disclosure relates to a foldable outer packaging for a packaged medical product, and a method for packaging a medical product using the foldable outer packaging.

### BACKGROUND

Medical products, such as bone implants, bone plates, intramedullary devices, artificial joints or similar items, are usually packaged in a controlled environment, in particular in a clean room, and are then sterilized before being transported to a customer, such as a hospital. As a rule, a double sterile barrier system is used for this. The double sterile barrier may consist of two plastic bags (primary packagings). The medical product is inserted into a first, inner plastic bag, which is vacuumed and sealed. While still in the controlled environment, the sealed first plastic bag is then placed in a second, outer plastic bag, which is also vacuumed and sealed. More than two plastic bags or just a single plastic bag may be used as a sterile barrier. The plastic bag(s) containing the medical product is/are then placed in an outer packaging box (secondary packaging), a cardboard box or something similar, for shipping and transportation.

The sterile barrier must remain intact to prevent contamination of the medical product. However, the plastic bag is dimensionally unstable. However, if the plastic bags are arranged loosely in a conventional (standardized) outer packaging box during transport, damage to the plastic bags may occur due to the plastic bags and medical products rubbing or hitting against each other, which impairs the guarantee of sterility. Large and heavy medical products in particular may damage the plastic bag if they move around in the secondary packaging and hit the inner walls of the secondary packaging. Therefore, additional protective components such as foams or other cushioning materials are usually provided, which add to the overall cost and complexity of the outer packaging system.

In order to avoid damage, the plastic bags are alternatively packaged in foldable individual packaging (secondary packaging), which in turn is arranged in the outer packaging box (tertiary packaging). For example, EP 2 739 238 B1 shows an outer packaging system for a medical device or a medical product with a sealed plastic bag as sterile primary packaging for the medical product and a foldable secondary packaging, which defines an inner space for holding the plastic bag.

Furthermore, EP 3 357 444 B1 discloses an outer packaging assembly for a medical device comprising at least two plastic bags. The outer packaging assembly further has a support element which, in an assembled configuration, is secured against movement relative to the interior of the at least two plastic bags. This securing is provided by the plastic bags themselves, which are folded around the support element. In addition, DE 100 14 419 A1, EP 3 789 314 A1, US 2010/0 320 112 A1, DE 698 13 860 T2 or DE 10 2005 038 491 B3 also show foldable outer packagings.

However, the known outer packagings are always dependent on the dimensions of the medical product and cannot be used flexibly in outer packaging boxes of different sizes. The existing solutions are also disadvantageous in terms of sustainability and the simplest possible separation of outer packaging materials during disposal, since they consist of different materials/composites (plastic/paper).

### SUMMARY

The objects and objectives of the disclosure are to eliminate or at least reduce the disadvantages of the prior art and, in particular, to provide a foldable outer packaging which enables the safe transport of sterile packaged medical products in standardized outer packaging boxes (tertiary packaging) with easy disposal.

Accordingly, the outer packaging (made, for example, from cardboard material) comprises a base plate, which is divided into three segments via two parallel spaced folding fold lines, a pivot plate, which is coupled to the base plate via a pivoting fold line aligned perpendicular to the folding fold lines, is also subdivided into three segments via the folding fold lines extending in a straight line into the pivot plate, and has a window-like cut-out or aperture in the middle of the three segments, and a side plate, which is coupled to the pivot plate via a turn-over fold line aligned parallel to the pivoting fold line, and is also divided into three segments via the folding fold lines that continue to extend in a straight line into the side plate.

According to the disclosure, the pivoting fold line extends over the entire length of the base plate and of the pivot plate.

Advantageous embodiments are explained below.

It may also be advantageous if the turn-over fold line extends over the entire length of the side plate and the pivot plate.

According to a particularly advantageous further development, the base plate, in particular the middle one of the three segments, may have a cutting pattern, preferably in the form of a plurality of longitudinal cuts. However, the cutting pattern may also be designed in any other way, for example in the form of a plurality of cross-sections.

According to an advantageous embodiment, the outer packaging may be made of a conventional packaging material, such as cardboard or paperboard.

Furthermore, the disclosure relates to a method of packaging a medical product using a foldable outer packaging according to the disclosure, comprising the following method steps:
placing a medical product on the middle one of the three segments of the base plate;
pivoting of the pivot plate along the pivoting fold line until the pivot plate rests on the base plate with the medical product protruding through the window-like cut-out;
turning over the side plate along the turn-over fold line to sandwich the base plate between the side plate and the pivot plate, and
folding over the two outer segments of the three segments of the base plate, pivot plate and side plate along the respective folding fold lines in the direction of the medical product.

Preferably, the medical product may be packaged in a sterile outer packaging before being placed on the middle one of the three segments of the base plate. The medical product may be packaged in a plastic bag as sterile outer packaging/sterile barrier/sterile barrier system/primary packaging. Alternatively, it is also conceivable that the medical product is packaged in two or more plastic bags, for example in the form of a double barrier system.

According to an advantageous further development, the outer packaging together with the medical product may be packaged in a, preferably standardized, outer packaging box after folding over the two outer segments of the base plate, pivot plate and side plate along the respective folding fold lines in the direction of the medical product. It is particularly advantageous if the outer packaging box is made of the same packaging material as the foldable outer packaging, for example cardboard or carton. Of course, other packaging materials are also conceivable, as long as the outer packaging and the outer packaging box are made of the same material.

According to the disclosure, it may also be conceivable that the window-like cut-out is adapted or adaptable to the dimensions of the medical product (e.g. by cutting out). For example, if the medical product has a rectangular cross-section or an elliptical cross-section, it may be useful if the cut-out is also rectangular or elliptical.

In other words, the disclosure relates to a blank of the outer packaging comprising the base plate, the pivot plate and the side plate, which are arranged in this order side by side and connected via the pivoting fold line and the turn-over fold line, and folding of the blank along the pivoting fold line and the turn-over fold line fixes a medical product placed on the base plate relative to the outer packaging. Preferably, the blank can also have a first side flap and a second side flap, which are connected to the pivoting plate and the base plate via the first folding fold line and the second folding fold line, so that when the side flaps are folded along the first folding fold line and the fourth folding fold line, a cavity is formed between the side flaps, into which the medical product protrudes. Preferably, the pivoting fold line and the turn-over fold line can be arranged parallel to each other, and the first folding fold line and the second folding fold line can each be arranged perpendicular to the pivoting fold line and the turn-over fold line. Particularly preferably, the side flaps and the base plate can fix the outer packaging in a, preferably standardized, outer packaging box (tertiary packaging).

In other words, the disclosure further relates to a method of packaging a medical product in a foldable outer packaging, preferably according to the invention, comprising the following steps:
placing the medical product on the base plate of the outer packaging;
folding the outer packaging along a pivoting fold line and a turn-over fold line to fix the medical product relative to the outer packaging; and
folding of the outer packaging along a first folding fold line and a second folding fold line to form a cavity into which the medical product protrudes.

### BRIEF DESCRIPTION OF THE DRAWINGS

The disclosure is explained in more detail below with reference to a preferred embodiment with the aid of Figures. The following is shown:

FIG. 1 shows a blank of an outer packaging according to a preferred embodiment;

FIG. 2 shows a first step for packaging a medical product with the outer packaging according to the preferred embodiment;

FIGS. 3 and 4 show a second step for packaging the medical product with the outer packaging according to the preferred embodiment;

FIGS. 5 and 6 show a third step for packaging the medical product with the outer packaging according to the preferred embodiment;

FIG. 7 shows a fourth step for packaging the medical product with the outer packaging according to the preferred embodiment;

FIG. 8 shows a step for packaging the medical product packaged with the outer packaging according to the preferred embodiment in a tertiary packaging;

FIG. 9 shows the medical product packaged in the tertiary packaging;

FIGS. 10 and 11 show medical products packaged in the tertiary packaging;

FIG. 12 shows a blank of an outer packaging according to a modification of the preferred embodiment;

FIG. 13 shows a medical product packaged in the outer packaging according to the modification of the preferred embodiment; and

FIG. 14 shows the medical product packaged in the outer packaging according to the modification of the preferred embodiment in the tertiary packaging.

The Figures are schematic in nature and are provided solely for the purpose of understanding the invention. Identical elements are marked with the same reference signs.

### DETAILED DESCRIPTION

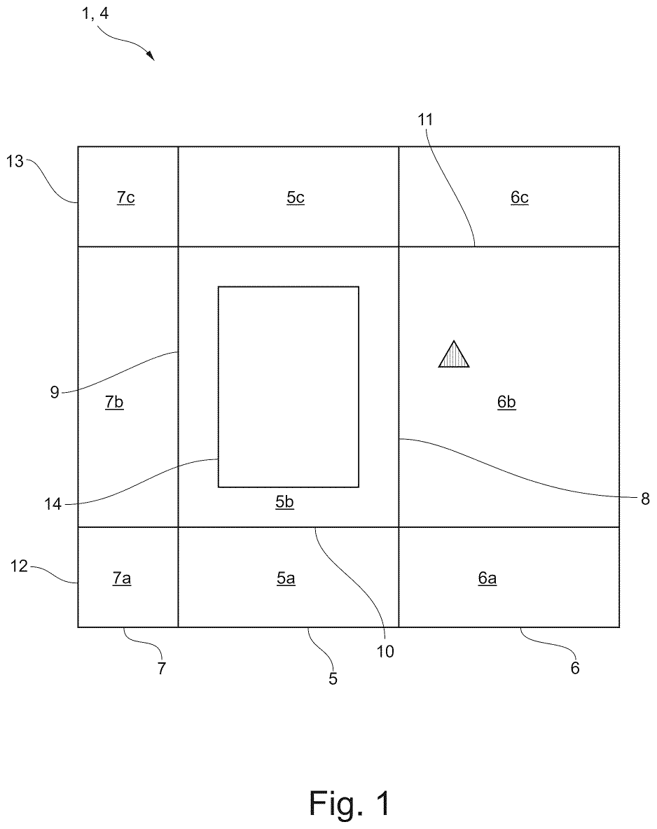

FIG. 1 shows a foldable (secondary) outer packaging 1 for a medical product 3 packaged in a sterile primary packaging/sterile barrier 2 in an unfolded state. In other words, FIG. 1 shows a blank or folding sheet 4 of the outer packaging 1 according to a preferred embodiment.

The outer packaging 1 according to the preferred embodiment has a pivot plate 5, a base plate 6 and a side plate 7, which are each connected to each other via a pivoting fold line 8 and a turn-over fold line 9. The pivot plate 5 is connected to the base plate 6 via a pivoting fold line 8 and is connected to the side plate 7 via a turn-over fold line 9. In the top view shown in FIG. 1, the side plate 7, the pivot plate 5 and the base plate 6 are therefore arranged next to each other and are each connected to each other via the turn-over fold line 9 or respectively via the pivoting fold line 8. The pivoting fold line 8 and the turn-over fold line 9 are arranged substantially parallel and divide the blank 4 (in FIG. 1 vertically) into three elements, i.e. the side plate 7, the pivot plate 5 and the base plate 6. In a plan view, as shown in FIG. 1, one surface of the pivot plate 5 is equal to one surface of the base plate 6, so that, as described in more detail below, the pivot plate 5, when folded along the pivoting fold line 8 over the base plate 6, completely covers the latter.

As shown in FIG. 1, the outer packaging 1 or respectively the blank 4 also has a first folding fold line 10 and a second folding fold line 11. The first folding fold line 10 and the second folding fold line 11 are arranged substantially perpendicular to the pivoting fold line 8 and the turn-over fold line 9. The blank 4 is thus subdivided into three (horizontal) segments in its direction of extension along the pivoting fold line 8 and the turn-over fold line 9. In other words, the pivot plate 5, the base plate 6 and the side plate 7 are each subdivided into three superimposed segments by the first folding fold line 10 and the second folding fold line 11. The pivot plate 5 is thus subdivided into a first, lower pivot-plate segment 5a, a second, middle pivot-plate segment 5b and a third, upper pivot-plate segment 5c. Similarly, the base plate 6 is subdivided by the first folding fold line 10 and the second folding fold line 11 into a first, lower base-plate segment 6a, a second, middle base-plate segment 6b and a third, upper base-plate segment 6c. The side plate 7 is subdivided by the first folding fold line 10 and the second folding fold line 11 into a first, lower side-plate segment 7a, a second, middle side-plate segment 7b and a third, upper side-plate segment 7c.

The first and third segments, i.e. the first pivot-plate segment 5a and the third pivot-plate segment 5c, the first base-plate segment 6a and the third base-plate segment 6c as well as the first side-plate segment 7a and the third side-plate segment 7c, are each the same size or have the same external dimensions. That is, in the plan view in FIG. 1, the horizontal distance between the lower, horizontal outer edge of the blank 4 and of the first folding fold line 10 is equal to the horizontal distance between the upper, horizontal outer edge of the blank 4 and of the second folding fold line 11.

As can be seen in FIG. 1, the blank 4 of the outer packaging 1 is subdivided by the fold lines 8, 9, 10, 11 into the nine segments 5a, 5b, 5c, 6a, 6b, 6c, 7a, 7b, 7c. The pivoting fold line 8 is arranged between the segments 5a, 5b, 5c, which form the pivot plate 5, and the segments 6a, 6b, 6c, which form the base plate 6. The turn-over fold line 9 is formed between the segments 5a, 5b, 5c of the pivot plate 5 and the segments 7a, 7b, 7c of the side plate 7. The first folding fold line 10 is formed between the segments 5a, 6a, 7a, which form a first side flap 12, and the segments 5b, 6b, 7b, whereas the second folding fold line 11 is arranged between the segments 5b, 6b, 7b and the segments 5c, 6c, 7c of a second side flap 13.

A cut-out 14 is formed in the pivot plate 5, in particular in the second, middle pivot plate segment 5b, as shown in FIG. 1. In the preferred embodiment, the cut-out 14 is shaped in the form of a rectangular passage opening. The shape and dimensions of the cut-out 14 are adapted to the medical product 3 to be packaged with the outer packaging 1, as described in more detail below. For example, the cut-out 14 may also be circular or elliptical or in the shape of any polygon.

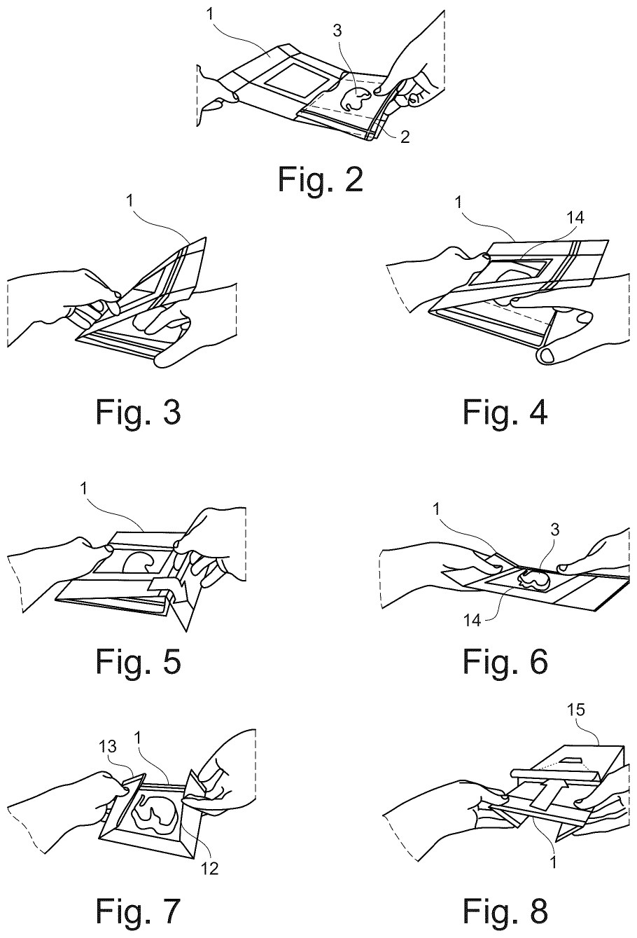

In order to package the medical product 3 with the outer packaging 1, the medical product 3, which is sterile-packed in the primary packaging 2, is first placed on the base plate 6, in particular the second, middle base-plate segment 6b, in a first step shown in FIG. 2. As shown in FIG. 2, the primary packaging 2 may protrude beyond the second base-plate segment 6b as long as the medical product 3 in the primary packaging 2 is placed on the second base-plate segment 6b. In other words, the base plate 6 serves as a receptacle for the medical product 3 packaged in the primary packaging 2.

In a second step, shown in FIGS. 3 and 4, the pivot plate 5 together with the side plate 7 is folded along the pivoting fold line 8 toward the base plate 6, so that the pivot plate 5 substantially covers the base plate 6. The medical product 3 in the primary packaging 2 protrudes through the cut-out 14 of the pivot plate 5. That is, the cut-out 14 embraces/encloses the medical product 3 in the folded state in order to fix it relative to the outer packaging 1.

Then, in a third step shown in FIGS. 5 and 6, the side plate 7 is then folded inward along the turn-over fold line 9 toward a lower side of the base plate 6 in order to hold the pivot plate 5 on the base plate 6. In other words, the side plate 7 is folded along the turn-over fold line 9 in order to sandwich the base plate 6 between itself and the pivot plate 5.

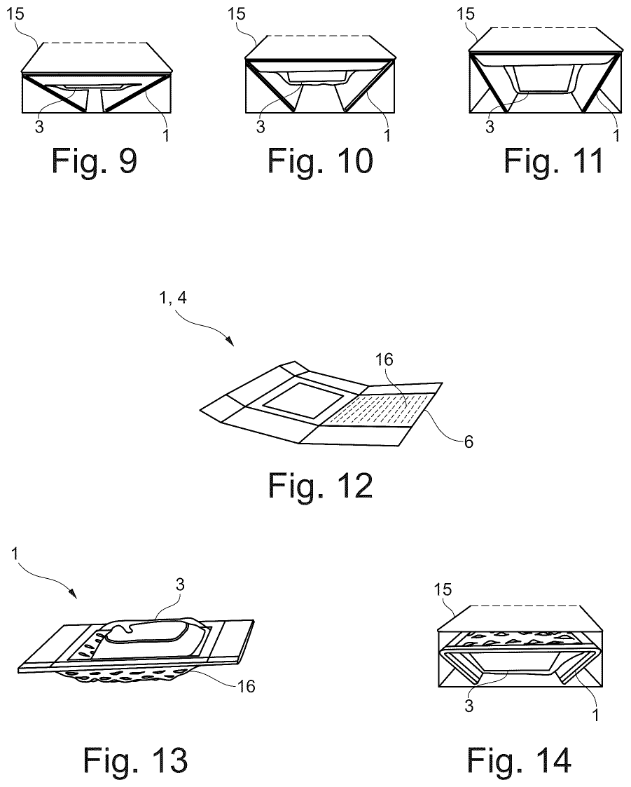

In a fourth step, shown in FIG. 7, the side flaps 12 and 13, i.e. the superimposed segments 5a, 6a, 7a as well as the superimposed segments 5b, 6b, 7b, are folded upward in the direction of the medical product 3 projecting through the cut-out 14. If the medical product 3 packaged in the outer packaging 1 is then placed in a, preferably standardized, outer packaging box (tertiary packaging) 15 for transport or shipping, for example, the side flaps 12, 13 fold outward again due to the inherent elasticity of the outer packaging material and in doing so come into contact with the inner surfaces of the outer packaging box 15. That is, as shown in FIG. 9, the outer packaging 1 according to the preferred embodiment lies in the folded state with the lower side of the base plate 6, i.e. the side facing away from the medical product 3, against a first inner surface of the outer packaging box 15. The edges of the side flaps 12, 13 of the outer packaging are in contact with a second inner surface of the outer packaging box 15 opposite the first inner surface, so that the outer packaging 1 is held in the outer packaging box 15. In other words, the outer packaging 1 spreads out in the outer packaging box 15.

As shown in FIG. 9, the side flaps 12, 13 form a cavity between them into which the medical product 3 can protrude. This means that the medical product 3 is fixed in the outer packaging box 15 by the outer packaging 1 without resting against one of the inner surfaces of the outer packaging box 15 or coming into contact with the outer packaging box 15. This prevents damage to the primary packaging 2 and the associated impairment of the sterility of the medical product 3.

The outer packaging 1 may also be used, as shown in FIGS. 10, 11, for medical products 3 of different sizes. If a large medical product 3 is packaged in the outer packaging 1 or in the outer packaging box 15, the side flaps 12, 13 are positioned more vertically compared to smaller medical products 3 and thus form a larger cavity between them.

In FIGS. 12 to 14, an outer packaging 1 according to a modification of the preferred embodiment is illustrated. Here, the base plate 6, in particular the second, middle base plate segment 6b, has a cutting pattern 16. According to the modification of the preferred embodiment, the cutting pattern 16 is in the form of a plurality of individual longitudinal cuts (arranged in rows) in the base plate 6. When the outer packaging 1 is folded according to the modification of the preferred embodiment and is placed in the outer packaging box 15, the cutting pattern 16 provides additional cushioning between the outer packaging box 15 and the medical product 3. In addition, the base plate 6, in particular its middle base-plate segment 6b, is given greater flexibility than an uncut plate due to the longitudinal cuts (longitudinal slits) provided therein, so that it can adapt to the outer shape of a product placed on it (see in particular FIG. 13).

In the preferred embodiment, the outer packaging 1 is made of a conventional outer packaging material, such as cardboard or carton. Since the outer packaging box 15 is also made of the conventional outer packaging material, it is no longer necessary to separate the outer packaging components during disposal. Of course, a different material may also be used for the outer packaging 1 and the outer packaging box 15, as long as the same material is used for the outer packaging components.

### List of Reference Signs

1 outer packaging
2 primary packaging
3 medical product
4 blank
5 pivot plate
5a first pivot-plate segment
5b second pivot-plate segment
5c third pivot-plate segment
6 base plate
6a first base-plate segment
6b second base-plate segment
6c third base-plate segment
7 side plate
7a first side-plate segment
7b second side-plate segment
7c third side-plate segment
8 pivoting fold line
9 turn-over fold line
10 first folding fold line
11 second folding fold line
12 first side flap
13 second side flap
14 cut-out
15 outer packaging box
16 cutting pattern

1.-6. (canceled)

7. An outer packaging for a medical product, the outer packaging being foldable and comprising:
a base plate divided into three base plate segments via folding fold lines, the folding fold lines being parallel and spaced;
a pivot plate; and
a side plate,
the pivot plate being coupled to the base plate via a pivoting fold line aligned perpendicular to the folding fold lines,
the pivot plate being subdivided into three pivot plate segments via the folding fold lines extending in a straight line into the pivot plate,
the pivot plate having a window-like cut-out or aperture in a middle portion of the three pivot plate segments,
the side plate being coupled to the pivot plate via a turn-over fold line aligned parallel to the pivoting fold line,
the side plate divided into three side plate segments via the folding fold lines that continue to extend in a straight line into the side plate,
the pivoting fold line extending over an entire length of the base plate and an entire length of the pivot plate, and
the base plate comprising a cutting pattern.

8. The outer packaging according to claim 7, wherein the turn-over fold line extends over an entire length of the side plate and the entire length of the pivot plate.

9. The outer packaging according to claim 7, wherein a middle base plate segment of the three base plate segments comprises the cutting pattern, the cutting pattern comprising a plurality of individual longitudinal cuts.

10. A method of packaging a medical product using the outer packaging according to claim 7, the method comprising the steps of:
placing the medical product on a middle base plate segment of the three base plate segments;
pivoting the pivot plate along the pivoting fold line until the pivot plate rests on the base plate with the medical product protruding through the window-like cut-out or aperture;
turning over the side plate along the turn-over fold line to sandwich the base plate between the side plate and the pivot plate; and
folding over two outer segments of the three base plate segments, two outer segments of the three pivot plate segments and two outer segments of the three side plate segments along the folding fold lines in a direction of the medical product.

11. The method according to claim 10, wherein the medical product is packaged in a sterile outer packaging before being placed on a middle base plate of the three base plate segments.

12. The method according to claim 10, wherein the outer packaging and the medical product are packaged in an outer packaging box after the step of folding over two outer segments of the three base plate segments, two outer segments of the three pivot plate segments and two outer segments of the three side plate segments along the folding fold lines in the direction of the medical product.
